(19) United States
(12) Patent Application Publication
(10) Pub. No.: US 2026/0166537 A1
(43) Pub. Date: Jun. 18, 2026

(54) METHOD FOR PREPARING CROWN ETHER-BASED COVALENT ORGANIC FRAMEWORKS AND APPLICATION IN IODINE ADSORPTION THEREOF

(71) Applicant: Chongqing University of Arts and Sciences, Chongqing City (CN)

(72) Inventors: Yuanzhe TANG, Chongqing City (CN); Qiangbin YANG, Chongqing City (CN); Yujuan JING, Chongqing City (CN); Zhonghao XU, Chongqing City (CN); Guangyong ZENG, Chongqing City (CN)

(21) Appl. No.: 19/530,988

(22) Filed: Feb. 5, 2026

Related U.S. Application Data
(63) Continuation of application No. PCT/CN2024/133744, filed on Nov. 22, 2024.

(30) Foreign Application Priority Data
Oct. 8, 2024 (CN) 202411389505.1

(51) Int. Cl. B01J 31/06; B01J 31/02; B01J 37/08; B01J 37/34; C07D 323/00

(52) U.S. Cl. CPC B01J 31/069; B01J 31/0204; B01J 37/08; B01J 37/343; C07D 323/00

(57) ABSTRACT

A method for preparing a crown ether-based covalent organic frameworks and its application in iodine adsorption are provided. According to the present disclosure, an 18-crown-6 ether group is introduced into the structure of the covalent organic framework (COF) material for iodine adsorption. The covalent organic frameworks form a one-dimensional pore channel structure in an orderly manner, and at the same time, the 18-crown-6 ether may selectively complex iodine molecules.

### CROSS-REFERENCE TO RELATED APPLICATIONS

This application is a continuation of International Patent Application No. PCT/CN2024/133744, filed on Nov. 22, 2024, and claims priority of Chinese Patent Application No. 202411389505.1, filed on Oct. 8, 2024. The contents of International Patent Application No. PCT/CN2024/133744 and Chinese Patent Application No. 202411389505.1 are hereby incorporated by reference.

### TECHNICAL FIELD

The present disclosure relates to a method for preparing a novel covalent organic framework having an 18-crown-6 ether group, targeting its application in iodine adsorption, and belongs to the field of adsorptive separation.

### BACKGROUND

In recent years, waste from used nuclear fuel generates volatile radioactive compounds containing iodine isotopes (¹²⁹I and ¹³¹I), which must be removed before the exhaust gases are discharged. Compared to conventional treatment processes used for adsorbing radioactive iodine and other iodine-containing compounds from exhaust gases, adsorption processes using porous solid adsorbents offer many advantages, including simple operation, low maintenance cost, and no need to use highly corrosive solutions. Currently, iodine adsorption applications of various adsorbents have been developed, including ceramics, zeolites, aerogels, metal-organic frameworks, and other materials. As an emerging type of adsorbent, covalent organic frameworks (COFs) are widely used due to their low density, high porosity, and excellent iodine adsorption capability. Research on iodine adsorption by COFs has found that electron-rich adsorbents may effectively adsorb electron-deficient iodine by forming complexes. To prepare electron-rich adsorbents, various synthetic strategies have been developed, including constructing π-π conjugated bonds, doping with a large number of heteroatoms, and incorporating different heterocyclic rings (such as triazine rings, pyrazine rings, etc.). Furthermore, nitrogen (N)-containing functional groups (such as amine, imine, imidazole, triazine, and pyridine) have been introduced into the framework structure to promote high iodine adsorption capacity.

Covalent organic frameworks (COFs) not only possess stable and tunable porous structures but also facilitate the design of functional group structures and properties. The crystallinity of COFs provides a well-ordered pore channel structure and designability of active sites within the framework, which benefits many applications such as catalysis, adsorption, and separation, including iodine adsorption. In principle, through designing suitable monomers or post-synthetic modification, COFs may possess adsorption active sites required for functionalization, thereby obtaining high iodine adsorption capacity. Therefore, there is a need to incorporate the 18-crown-6 ether group into the pore channel structure of COFs while maintaining high crystallinity and high porosity. Enabling the COF to simultaneously possess a one-dimensional pore channel structure and the 18-crown-6 ether groups capable of complexing iodine may significantly improve the iodine adsorption performance of the COF.

### SUMMARY

The objective of the present disclosure is to provide a method for preparing a novel covalent organic frameworks having an 18-crown-6 ether group and its application in iodine adsorption. The present disclosure prepares a covalent organic framework (COF)-4,4′,4″,4″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6) material. The prepared COF-DB18C6 possesses both a macroporous structure and properties of strong interaction force. The prepared COF-DB18C6 adsorbent not only has advantages such as high stability, simple preparation process, mild synthesis conditions, high efficiency, and rapidness, but also this COF-DB18C6 exhibits very high adsorption performance and regeneration performance in iodine vapor.

The technical scheme of the present disclosure is as follows.

A novel COF-DB18C6 having an 18-crown-6 ether group is specifically prepared according to the following method:
in a 100 milliliters (mL) reaction vessel, placing 2,5-dimethylpyrazine (DMP), 4,4′,4″,4′″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6), sodium hydroxide, trimethylbenzene, and methanol in the reaction vessel, ultrasonically treating for a period until dissolution, subsequently, heating a reaction system in an oven for a period, after the reaction is completed, performing cooling and centrifugation; washing several times with N,N-dimethylformamide (DMF), tetrahydrofuran (THF), water, and ethanol, and drying under vacuum at 100 degrees Celsius (° C.) to obtain a pale yellow COF-DB18C6;
the mixed volume of trimethylbenzene and methanol is 50-80 mL;
the volume ratio of trimethylbenzene to methanol is 1:0.5-10;
the molar ratio of DMP to DB18C6 is 1:1-10;
the concentration of DB18C6 dissolved in the mixed solution is 0.01-0.05 moles per liter (mol/L);
the concentration of sodium hydroxide in the mixed solution is 1-50 milligrams per milliliter (mg/mL);
the reaction temperature is 80° C.-200° C.; and
the reaction time is 0.5-7 days.

Compared with the prior art, the substantive advantages of the present disclosure are as follow.

The preparation process is simple and suitable for large-scale preparation.

The synthesis conditions are mild and easy to prepare in a conventional synthesis environment.

COF-DB18C6 has high chemical stability and thermal stability, making it suitable for iodine adsorption under different harsh conditions.

COF-DB18C6 has high adsorption performance for iodine and is capable of addressing issues related to radioactive accidents, environmental pollution and other issues.

### BRIEF DESCRIPTION OF THE DRAWINGS

FIG. 1 is a Brunauer-Emmett-Teller (BET) schematic diagram of the covalent organic framework (COF)-4,4′,4″,4′″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6) according to Embodiment 1 of the present disclosure.

FIG. 2 is an X-ray diffraction (XRD) schematic diagram of the COF-DB18C6 according to Embodiment 1 of the present disclosure.

FIG. 3 is a thermogravimetric analysis (TGA) schematic diagram of the COF-DB18C6 according to Embodiment 1 of the present disclosure.

FIG. 4 is a schematic diagram showing the iodine adsorption kinetic performance of COF-DB18C6 according to Embodiment 1 of the present disclosure.

FIG. 5 is a test chart showing the cyclic performance of COF-DB18C6 for iodine vapor according to Embodiment 1 of the present disclosure.

FIG. 6 is the flowchart of the preparation method of COF-DB18C6 according to an embodiment of the present disclosure.

### DETAILED DESCRIPTION OF THE EMBODIMENTS

The present disclosure is described in detail below with reference to specific embodiments. However, the present disclosure is not limited to the following embodiments. All variations implemented without departing from the content and scope of the present disclosure shall be included within the technical scope of the present disclosure.

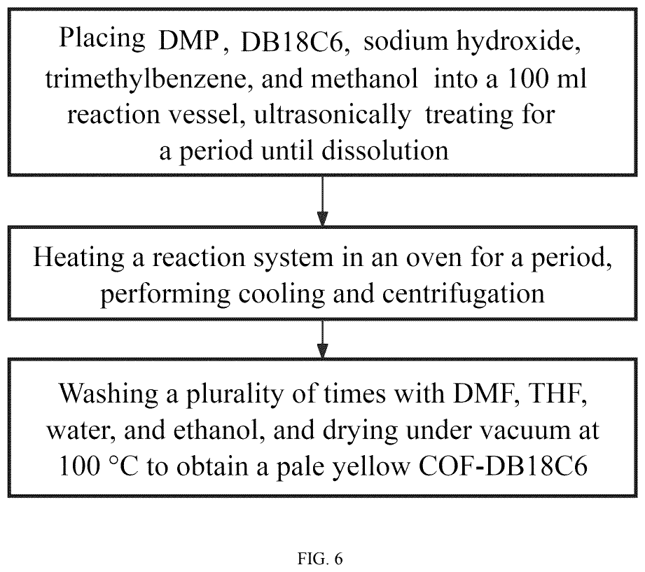

### Embodiment 1: Preparation of Covalent Organic Framework (COF)-4,4′,4″,4″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6) (as Shown in FIG. 6)

In a 100 milliliters (mL) reaction vessel, 2,5-dimethylpyrazine (DMP) (1 millimole (mmol), 108.2 milligrams (mg)), 4,4′,4″,4′″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6) (0.5 mmol, 310.2 mg), sodium hydroxide (140 mg), trimethylbenzene (30 mL), and methanol (30 mL) are placed into the reaction vessel, and ultrasonicated for a period until dissolution. Subsequently, the reaction system is reacted in an oven at 180 degrees Celsius (° C.) for 4 days. After the reaction is completed, the mixture is cooled and centrifuged, and then washed 3 times with N,N-dimethylformamide (DMF), 3 times with tetrahydrofuran (THF), 3 times with water, and 3 times with ethanol. Finally, it is dried under vacuum at 100° C. to obtain a pale yellow COF-DB18C6.

FIG. 6 illustrates a schematic diagram of preparation of the COF-DB18C6 prepared in Embodiment 1 of the disclosure.

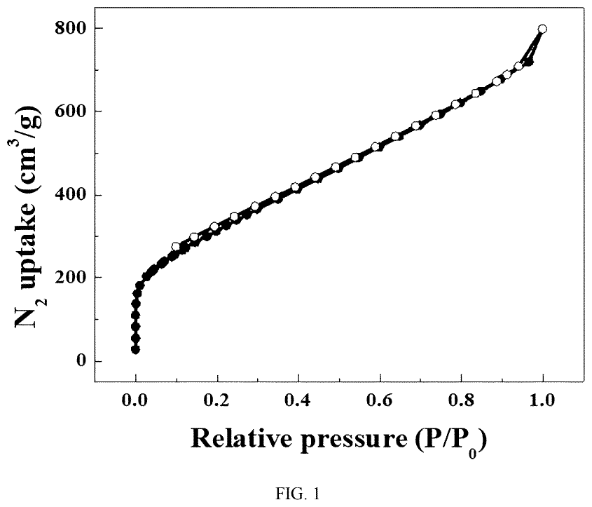

FIG. 1 illustrates a schematic diagram of BET analysis of the COF-DB18C6 prepared in Embodiment 1 of the disclosure. As shown in FIG. 1, the COF-DB18C6 possesses a high specific surface area.

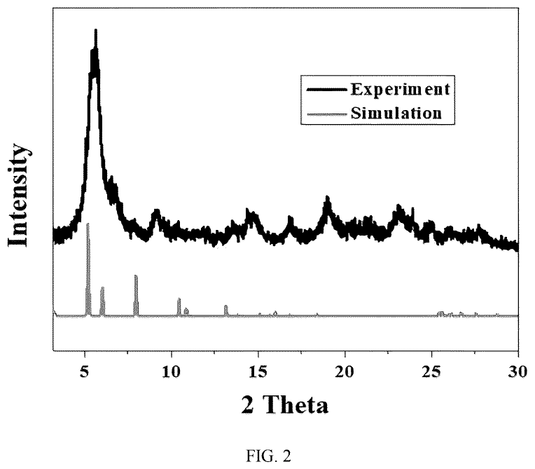

FIG. 2 shows a schematic diagram of XRD spectra of the COF-DB18C6 prepared in Embodiment 1 of the disclosure. As shown in FIG. 2, the COF-DB18C6 displays a high crystallinity.

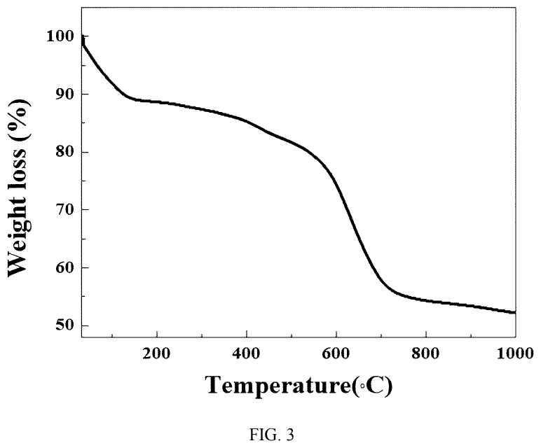

FIG. 3 illustrates a schematic diagram of a TGA curve of the COF-DB18C6 prepared in Embodiment 1 of the disclosure. As shown in FIG. 3, the COF-DB18C6 exhibits high thermal stability.

### Embodiment 2: Preparation of COF-DB18C6

In a 100 mL reaction vessel, DMP (1 mmol, 108.2 mg), DB18C6 (0.5 mmol, 310.2 mg), sodium hydroxide (140 mg), trimethylbenzene (40 mL), and methanol (40 mL) are placed into the reaction vessel, and ultrasonicated for a period until dissolution. Subsequently, the reaction system is reacted in an oven at 180° C. for 4 days. After the reaction is completed, the mixture is cooled and centrifuged, and then washed 3 times with DMF, 3 times with THE, 3 times with water, and 3 times with ethanol. Finally, it is dried under vacuum at 100° C. to obtain a pale yellow COF-DB18C6.

### Embodiment 3: Preparation of COF-DB18C6

In a 100 mL reaction vessel, DMP (1 mmol, 108.2 mg), DB18C6 (0.5 mmol, 310.2 mg), sodium hydroxide (140 mg), trimethylbenzene (20 mL), and methanol (40 mL) are placed into the reaction vessel, and ultrasonicated for a period until dissolution. Subsequently, the reaction system is reacted in an oven at 180° C. for 4 days. After the reaction is completed, the mixture is cooled and centrifuged, and then washed 3 times with DMF, 3 times with THF, 3 times with water, and 3 times with ethanol. Finally, it is dried under vacuum at 100° C. to obtain a pale yellow COF-DB18C6.

### Embodiment 4: Preparation of COF-DB18C6

In a 100 mL reaction vessel, DMP (0.5 mmol, 54.1 mg), DB18C6 (0.5 mmol, 310.2 mg), sodium hydroxide (140 mg), trimethylbenzene (30 mL), and methanol (30 mL) are placed into the reaction vessel, and ultrasonicated for a period until dissolution. Subsequently, the reaction system is reacted in an oven at 180° C. for 4 days. After the reaction is completed, the mixture is cooled and centrifuged, and then washed 3 times with DMF, 3 times with THF, 3 times with water, and 3 times with ethanol. Finally, it is dried under vacuum at 100° C. to obtain a pale yellow COF-DB18C6.

### Embodiment 5: Preparation of COF-DB18C6

In a 100 mL reaction vessel, DMP (1 mmol, 108.2 mg), DB18C6 (1 mmol, 620.4 mg), sodium hydroxide (140 mg), trimethylbenzene (30 mL), and methanol (30 mL) are placed into the reaction vessel, and ultrasonicated for a period until dissolution. Subsequently, the reaction system is reacted in an oven at 180° C. for 4 days. After the reaction is completed, the mixture is cooled and centrifuged, and then washed 3 times with DMF, 3 times with THE, 3 times with water, and 3 times with ethanol. Finally, it is dried under vacuum at 100° C. to obtain a pale yellow COF-DB18C6.

### Embodiment 6: Preparation of COF-DB18C6

In a 100 mL reaction vessel, DMP (1 mmol, 108.2 mg), DB18C6 (0.5 mmol, 310.2 mg), sodium hydroxide (350 mg), trimethylbenzene (30 mL), and methanol (30 mL) are placed into the reaction vessel, and ultrasonicated for a period until dissolution. Subsequently, the reaction system is reacted in an oven at 180° C. for 4 days. After the reaction is completed, the mixture is cooled and centrifuged, and then washed 3 times with DMF, 3 times with THE, 3 times with water, and 3 times with ethanol. Finally, it is dried under vacuum at 100° C. to obtain a pale yellow COF-DB18C6.

### Embodiment 7: Preparation of COF-DB18C6

In a 100 mL reaction vessel, DMP (1 mmol, 108.2 mg), DB18C6 (0.5 mmol, 310.2 mg), sodium hydroxide (140 mg), trimethylbenzene (30 mL), and methanol (30 mL) are placed into the reaction vessel, and ultrasonicated for a period until dissolution. Subsequently, the reaction system is reacted in an oven at 150° C. for 4 days. After the reaction is completed, the mixture is cooled and centrifuged, and washed 3 times with DMF, 3 times with THF, 3 times with water, and 3 times with ethanol. It is then dried under vacuum at 100° C. to obtain a pale yellow COF-DB18C6.

### Embodiment 8: Preparation of COF-DB18C6

In a 100 mL reaction vessel, DMP (1 mmol, 108.2 mg), DB18C6 (0.5 mmol, 310.2 mg), sodium hydroxide (140 mg), trimethylbenzene (30 mL), and methanol (30 mL) are placed into the reaction vessel, and ultrasonicated for a period until dissolution. Subsequently, the reaction system is reacted in an oven at 180° C. for 2 days. After the reaction is completed, the mixture is cooled and centrifuged, and then washed 3 times with DMF, 3 times with THE, 3 times with water, and 3 times with ethanol. Finally, it is dried under vacuum at 100° C. to obtain a pale yellow COF-DB18C6.

### Embodiment 9: Adsorption of Iodine Vapor by COF-DB18C6

Before the experiment, all COF-DB18C6 samples from Embodiment 1, Embodiment 2, Embodiment 3, Embodiment 4, Embodiment 5, Embodiment 6, Embodiment 7, Embodiment 8 and the glass containers are fully degassed and dried under vacuum. Then, 15 mg of COF-DB18C6 and an excess amount of iodine are added into each of two 20 mL sample vials, respectively. Both sample vials are placed simultaneously into a sealable glass container. After sealing, the containers are placed in an oven at 75° C. to heat. They are taken out at certain time intervals. After cooling to room temperature, the mass change of COF-DB18C6 before and after iodine adsorption are weighed.

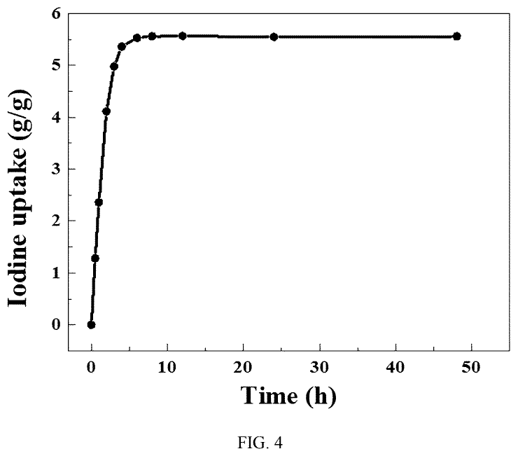

FIG. 4 illustrates a schematic diagram of iodine-adsorption kinetic performance of the COF-DB18C6 prepared in Embodiment 1 of the disclosure. As shown in FIG. 4, the iodine uptake of the COF-DB18C6 increases linearly within the first 3 hours and reaches adsorption saturation in approximately 6 hours. This indicates that the COF-DB18C6 of Embodiment 1 possesses very high adsorption performance and fast adsorption rate for iodine, with an iodine uptake reaching 5.56 g/g. The disclosure not only provides a novel 18-crown-6 ether group-based covalent organic framework but also achieves high performance in iodine adsorption. As shown in Table 1, the iodine uptake of the COF-DB18C6 from Embodiment 1, Embodiment 2, Embodiment 3, Embodiment 4, Embodiment 5, Embodiment 6, Embodiment 7, Embodiment 8 is shown.

TABLE 1
Iodine adsorption capacity of COF-DB18C6 from
Embodiment 1, Embodiment 2, Embodiment 3, Embodiment
4, Embodiment 5, Embodiment 6, Embodiment 7,
Embodiment 8 for iodine adsorption

| | Iodine adsorption capacity |
|---|---|
| Material name | (grams per gram (g/g)) |
| Embodiment 1: COF-DB18C6 | 5.56 |
| Embodiment 2: COF-DB18C6 | 2.83 |
| Embodiment 3: COF-DB18C6 | 4.21 |
| Embodiment 4: COF-DB18C6 | 3.78 |
| Embodiment 5: COF-DB18C6 | 2.70 |
| Embodiment 6: COF-DB18C6 | 3.52 |
| Embodiment 7: COF-DB18C6 | 3.11 |
| Embodiment 8: COF-DB18C6 | 4.18 |

### Embodiment 10: Cyclic Performance Test of Regenerated COF-DB1806 Adsorbent for Iodine Vapor Adsorption

After the COF-DB18C6 from Embodiment 1 is selected for the iodine adsorption test, 200 mg of the iodine vapor-adsorbed COF-DB18C6 (denoted as I₂-COF-DB18C6) is taken and stirred with 500 mL of ethanol at room temperature for 12 hours. It is washed 3 times, filtered, and taken out, followed by drying under vacuum at 100° C. Then the weight percentage change of COF-DB18C6 before and after iodine desorption are determined. The desorbed material is reused for the iodine adsorption experiment, and the cycle is repeated for at least 5 times.

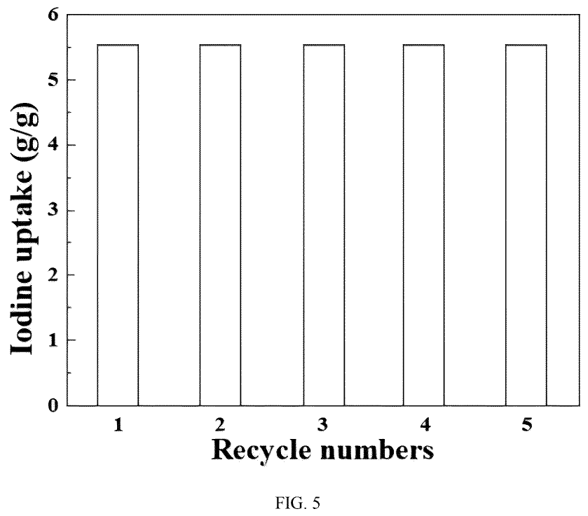

As shown in FIG. 5, after 5 cycles of adsorption in iodine vapor, the maximum adsorption capacity of COF-DB18C6 still maintain more than 99% of its original maximum adsorption capacity.

Certainly, the above specific embodiments are only detailed explanations of the corresponding technical schemes of the present disclosure and are not limited to the above implementation modes. For those skilled in the art, various changes or modifications in different forms may still be made based on the above embodiments. Not all implementation modes are listed here. Any variations or modifications derived from principles or mechanisms similar to the above are within the protection scope of the present disclosure.

What is claimed is:

1. A novel covalent organic framework (COF)-DB18C6 having an 18-crown-6 ether group, wherein the novel covalent organic framework having the 18-crown-6 ether group is prepared by a method comprising:
placing 2,5-dimethylpyrazine, 4,4′,4″,4″-(6,7,9,10,17,18,20,21-octahydrodibenzo[B,K][1,4,7,10,13,16]hexaoxacyclooctadecine-2,3,13,14-tetrayl)tetrabenzaldehyde (DB18C6), sodium hydroxide, trimethylbenzene, and methanol into a 100 milliliters (mL) reaction vessel, ultrasonically treating for a period until dissolution, subsequently heating a reaction system in an oven for a period; after a reaction is completed, performing cooling and centrifugation; washing a plurality of times with N,N-dimethylformamide (DMF), tetrahydrofuran (THF), water, and ethanol, and drying under vacuum at 100 degrees Celsius (° C.) to obtain a pale yellow COF-DB18C6.

2. The novel COF-DB18C6 having the 18-crown-6 ether group according to claim 1, wherein a mixed volume of the trimethylbenzene and the methanol is 50-80 mL.

3. The novel COF-DB18C6 having the 18-crown-6 ether group according to claim 1, wherein a volume ratio of the trimethylbenzene to the methanol is 1:0.5-10.

4. The novel COF-DB18C6 having the 18-crown-6 ether group according to claim 1, wherein a molar ratio of the 2,5-dimethylpyrazine to the DB18C6 is 1:1-10.

5. The novel COF-DB18C6 having the 18-crown-6 ether group according to claim 1, wherein a concentration of the DB18C6 dissolved in a mixed solution is 0.01-0.05 moles per liter (mol/L).

6. The novel COF-DB18C6 having the 18-crown-6 ether group according to claim 1, wherein a concentration of the sodium hydroxide in a mixed solution is 1-50 milligrams per milliliter (mg/mL).

7. The novel COF-DB18C6 having the 18-crown-6 ether group according to claim 1, wherein a reaction temperature is 80° C.-200° C.

8. The novel COF-DB18C6 having the 18-crown-6 ether group according to claim 1, wherein a reaction time is 0.5-7 days.
